(19) United States
(12) Patent Application Publication
(10) Pub. No.: US 2016/0278828 A1
(43) Pub. Date: Sep. 29, 2016

(54) OSTEOSYNTHESIS PLATE FOR DISTAL FIBULA / LATERAL MALLEOULUS

(71) Applicant: Umberto Ragghianti, San Lorenzo Di Moriano (IT)

(72) Inventor: UMBERTO RAGGHIANTI, SAN LORENZO DI MORIANO (IT)

(21) Appl. No.: 15/035,907

(22) PCT Filed: Nov. 10, 2014
(86) PCT No.: PCT/IB2014/065919

§ 371 Date: May 11, 2016

(30) Foreign Application Priority Data
Nov. 11, 2013 (IT) PI2013A000095

(51) Int. Cl. A61B 17/80

(52) U.S. Cl. CPC A61B 17/8061

(57) ABSTRACT

An osteosynthesis plate suitable for the fixation of fractures of the distal fibula/lateral malleolus includes a twisted distal portion and a proximal portion with a constant orientation that is perfectly congruent with the actual geometry of the distal fibula and it can be positioned with a rear orientation in the distal region. An osteosynthesis plate according to the invention favours, among other things, the restoration of the length and bone rotation and the peroneal-talar joint congruence, decreases tissue sufferance and facilitates the positioning of syndesmosis screws.

### TECHNICAL FIELD

The present invention relates to a plate for the osteosynthesis of the distal fibula/lateral malleoulus.

### STATE OF THE ART

It is widely known in the art the use of plates and relating screws for fixation of bone fractures. In particular, for the synthesis of long bones the type of plate, P, most commonly used is, as shown in FIG. 1a and 1b, a rectilinear finger bar and arcuate in the direction orthogonal to the longitudinal one, usually made of metal, in which holes are present, F, circular and/or slot-shaped through which bone screws are placed. These plates are usually provided in various lengths and with a variable number of aligned holes as a function of the length.

Some variants of the common bone plates require changes to the configuration of the holes that facilitate compression of the fragments of the fracture when the screws are inserted in place. Another variation on the configuration of the holes is the presence of female threads in the inner perimeter of the holes in which are screwed corresponding male threads provided at the head of the bone screw. In this way, when the screw is positioned in the hole and screwed into the bone, this also becomes integral to the plate and rotation of the plate itself is so prevented.

Variants of common bone plates have been designed in particular for the fixation of fractures of the malleolus, specifically to take into account the geometry of the bone, and the fact that the fracture is located near an end area of a long bone.

For example, in FIG. 2 it is shown in the coronal plane the group tibia-fibula, a plate, P′, for osteosynthesis is mounted laterally onto the fibula for the fixation of a fracture of the lateral malleoulus. The plate, in the longitudinal direction, follows a curvilinear path designed to follow the lateral profile of the fibula in the distal and ankle region. It is also visible in the figure the head of the screws, V, positioned in each hole of the plate P′.

Another known variant is shown in FIG. 3, in which the distal end, D, of the plate P″ has a triangular shape widened with respect to the elongated body, C, of the plate and it lies on a plane parallel to that in which lies the body C of the same plate, the two portions being connected by an inclined connection flat portion, I, suitably rounded. At the distal region, the holes, F, instead of being arranged aligned longitudinally are arranged in a triangle. Obviously, with all the above outlined types of plates P, P′, P″, each of the above hole configuration may be provided.

Plates similar to that described above are disclosed in US patent application US 2008/300637 in which at FIG. 23 is shown a plate similar to that of FIG. 3 attached to this description, which is applied to the distal fibula for the fixation of a peroneal/malleolar fracture. In the subsequent FIGS. 24 to 28 are shown some variants of the plate of FIG. 23 which take into account the increased thickness of the fibula in the malleolar region, but does not take into account the actual conformation of the bone surfaces in such distal region of the fibula, therefore causing problems of application and fastening to the bone.

Another type of plate suitable for the fixation of fractures of the distal fibula is shown in the same US patent application US 2008/300637 at FIGS. 31 to 38. As shown in FIG. 31, the plate is suitable to be applied to a distal region of the fibula starting from a zone immediately above the malleoulus, and as shown in FIGS. 37 and 38 it has a slight twisting, similar to a small horn facing down, in correspondence of an end of one side, groped to better follow the contour of the bone. However, this solution is not designed for the malleolar region of the fibula but for a zone immediately above this, and it does not allow faithfully following the contour of the bone and therefore it gives problems when it is applied and fastened to the bone. More exactly, the latter type of plate, as well as that previously described, is not able to follow the conformation of the bone face on which the peroneal muscles and tendons lie.

A further variant of the osteosynthesis plate, specifically suitable to be applied at one end of bone, is described in US patent application US 2011/0213420 A1, in which it is disclosed a contoured plate provided at one end with two hook members suitable to penetrate the bone at one end of the latter.

All variants of plates for osteosynthesis mentioned above may be used for the fixation of fractures of the lateral malleolus and one comes from time to time preferred to the other depending on the type and location of the fracture, the presence or absence of breakage of the syndesmosis and other factors.

In most cases, the osteosynthesis plate is applied in a lateral position, as shown in FIG. 2, while in some cases it is necessary to use a rear positioning. In any case, all plates for osteosynthesis mentioned have limitations because they do not take into consideration the actual geometry of the lateral malleoulus and the distal fibula.

In FIG. 4, it is shown in the coronal plane the distal portion of the group consisting of the tibia T, and fibula, R, in which a fracture line is shown at malleoulus, M, level. In the side view of FIG. 5, and in the three sectional views made at different heights, of FIGS. 6, 7 and 8, it is possible to observe the geometry of the distal fibula. In the most distal portion, corresponding with the malleolar region, the fibula R has a first face, S1, substantially flat and oriented predominantly towards a rear direction, and a second face, S2, also substantially flat but mostly oriented towards a lateral direction, and then substantially orthogonal to the previous one. More precisely, at malleolar level, corresponding approximately to the section plane VI-VI, the first face S1 forms an angle of about 20° relative to a back plane, while the second face S2 forms an angle of about 25° with respect to a side plane. The first and second faces S1 and S2 are separated by a crest of the bone, N1, which is rather rounded in the most distal region (FIG. 6) while it has a much more sharp edge in a region immediately above the malleolar region (FIG. 7). The crest N1, which is the site of insertion of the fibrous septum which distinguishes and separates the anterior compartment of the leg from the lateral compartment of the leg, and it is of fundamental importance for the proper restoration of the rotation of the distal fragment on the proximal fragment as it forms the anatomical structure more easily and effectively identified as fixation repere. The bone crest N1, which at distal level is directed substantially posteriorly, rotates towards the front side while it goes up until it joins with an anterior bone crest, N2. This results in a conformation of the distal end of the fibula in which the first face S1 is twisted, facing mainly back at the most distal region and running substantially laterally oriented in the most proximal region. At the distal end of the fibula, there is also a rear bone crest, N3, which delimits on one side the first face S1 and which runs in a substantially vertical direction.

The bone plates of the known art briefly described above are mainly applied on the lateral side so in the most distal region they are superimposed on the second side S2, while in their proximal region they are superimposed on the first face S1. The presence of the bone crest N1 prevents the straight plates, as the plate P of FIG. 1, from adhering properly to the faces S1 and S2. Shaped bone plates, such as plate P′ of FIG. 2, plate P″ of FIG. 3 and FIGS. 23 to 28 of the cited application US 2008/300637 or even the plate described in the aforementioned US application US 2011/0213420 A1 attempt to overcome this problem, but not succeeding in an efficient manner due to the geometry of the first and second faces S1 and S2 and the bone crest N1 that separates them.

Twisted osteosynthesis plates are also known as the one described in US patent application US 2004/116930, and shown to FIGS. 43 to 48 therein. The plate for osteosynthesis shown is twisted only in correspondence of an end portion of a length of 1 or 2 cm and is suitable for the fixation of fractures of the ulna. In general, in the aforementioned US application it is generically cited the possibility to align the plate for osteosynthesis in order to allow the inner surface of the plate to follow the three-dimensional contour of a bone, for the length of the plate. It mentions the fact that the plates may include internal surfaces curved, bent, warped, and/or tubular that fit to the bone face to guide the plate, to improve the fixation of the fracture, and further to increase the stability, improving the gripping of bone fragments. However, the cited US document does not suggest what the best way is to shape a plate for osteosynthesis that should be applied to the distal fibula including the malleolar region in order to fix fractures of the distal fibula/lateral malleolus.

In addition, conventional procedures for fixing fractures of the distal fibula/lateral malleolus are not optimal and can be optimized thanks to the use of peculiar osteosynthesis plates.

In fact, when using a conventional osteosynthesis plate, it is first performed the fracture fixation as precisely as possible, trying to reconstruct the correct morphology and peroneal-talar joint matching, after which the osteosynthesis plate is temporarily stabilized in a lateral position by using fixation clamps, such as a Weber or Verbrugge forceps, and finally it is made integral to the bone fragments by the application of the relevant bone screws. According to the conventional techniques of lateral positioning plates of conventional type, during the steps of stabilization and fastening of the plate there is a strong risk to decompose again the fracture due to the non-perfect matching of the geometry of the plate with the bone geometry.

### SUMMARY OF THE INVENTION

An object of the present invention is then to propose an osteosynthesis plate for application to a bone wall belonging to the distal fibula and the lateral malleolus in order to fix fractures of the distal fibula/lateral malleolus, able to overcome the limitations of the above plates of the prior art.

Further object of the present invention is to propose an osteosynthesis plate with optimized shape to fit faithfully to the geometry of the distal fibula/lateral malleolus so that the bone contact surface of the plate adheres to the surface of the bone for the entire length.

Another object of the invention is to propose a plate for the fixation of fractures at the distal fibula/lateral malleolus that allows using, in the distal area, screws longer than those used in the known art.

Another object of the present invention is to propose a plate for the fixation of fractures of the distal fibula/lateral malleolus that allows placing a syndesmosis screw in one of the holes of the plate itself.

Another object of the present invention is to propose a plate for the fixation of fractures of the distal fibula/lateral malleolus able to reduce the risk of wound dehiscence in the distal region.

Another object of the present invention is to propose a plate for the fixation of fractures of the distal fibula/lateral malleolus able to forewords push the distal fragment usually moved backward because of the fracture, thus restoring the original length and bone rotation and the tibio-peroneus-talar matching.

According to one aspect of the present invention, the above objects and others are achieved by means of an osteosynthesis plate according to what is expressed and characterized in the independent claim 1.

The claims dependent on claim 1 describe other characteristics of the osteosynthesis plate of the present invention or variants to the main inventive idea.

Conventionally, a plate for osteosynthesis of the distal fibula and the lateral malleolus comprises an elongated body provided with a distal end, a proximal end, an upper surface, a lower surface determining a longitudinal axis and a plurality of through holes from the upper surface to the lower surface disposed substantially along said longitudinal axis.

According to the present invention the said elongated body comprises a distal portion geometrically twisted so as to faithfully match the conformation of a first face of the distal end of the fibula oriented predominantly towards a rear direction at malleolar level and predominantly laterally at a distal region coming just over said malleolar level, so that bone contact portions of the plate with the bone surface remain in contact with said first bone face for the entire length of the plate.

Advantageously, the total angle of twist between the above-mentioned distal end and the other end of the said distal portion is between 40° and 80°.

The twisted distal portion extends from the distal end for a length between 30 mm and 70 mm, while a proximal portion extending contiguous to it without solution of continuity of the relative upper and lower surfaces has a profile with constant orientation.

Still advantageously, the distal end is chamfered on both sides with a chamfer length of at least 2 mm.

Still advantageously, said elongated body has a substantially constant section with a profile of the upper and lower surfaces arcuate in the transverse direction, except in a distal area of a length not exceeding 30 mm in which the profile of the upper and lower surfaces is substantially flat.

A plate for osteosynthesis of the lateral malleolus as outlined above can be applied posteriorly oriented in the most distal region of the malleolus and following the shape of a substantially flat surface of the same it turns in a lateral oriented position in a more proximal region of the latter. This positioning matches the anatomy of the malleoulus and in general the distal fibula, facilitating and enhancing the restoration from the fracture.

### BRIEF DESCRIPTION OF DRAWINGS

These and other advantages associated with the device of the invention will become more easily understood through the illustration of preferred embodiments, non-limiting, as described below with the aid of the enclosed drawings, in which:

FIGS. 1 to 3 represent plates for osteosynthesis of the prior art: FIG. 1 a shows a front view of a first plate for osteosynthesis of the prior art; FIG. 1b shows a top view of the plate of FIG. 1a; FIG. 2 shows a second plate for osteosynthesis of the prior art whose lateral profile is visible, laterally fastened by using proper screws to a distal portion of a right fibula viewed in the coronal plane together with its shinbone; FIG. 3 shows a perspective view of a third plate for osteosynthesis of the prior art;

FIG. 4 shows a view in the coronal plane of the distal region of right shinbone and fibula group in which it is shown a fracture zone at malleoulus level;

FIG. 5 shows a side view from the outer side of the distal portion of a right shinbone and fibula group;

FIGS. 6 to 8 show the profiles in section of the fibula at different heights obtained respectively according to the planes VI-VI, VII-VII and VIII-VIII of FIG. 5;

FIG. 9 shows a front view of a plate for osteosynthesis of the present invention;

FIG. 10 shows a sectional view taken along the section line X-X of FIG. 9;

FIG. 11 shows a side view of the plate for osteosynthesis of FIG. 9;

FIG. 12 shows a bottom view of the plate of FIG. 9;

FIG. 13 schematically shows the profile of a distal end and a proximal end of the plate for osteosynthesis of FIG. 9;

FIG. 14 shows a variant embodiment of a plate for osteosynthesis according to the present invention applied to the distal portion of a right fibula in view in the coronal plane together with a shinbone thereof.

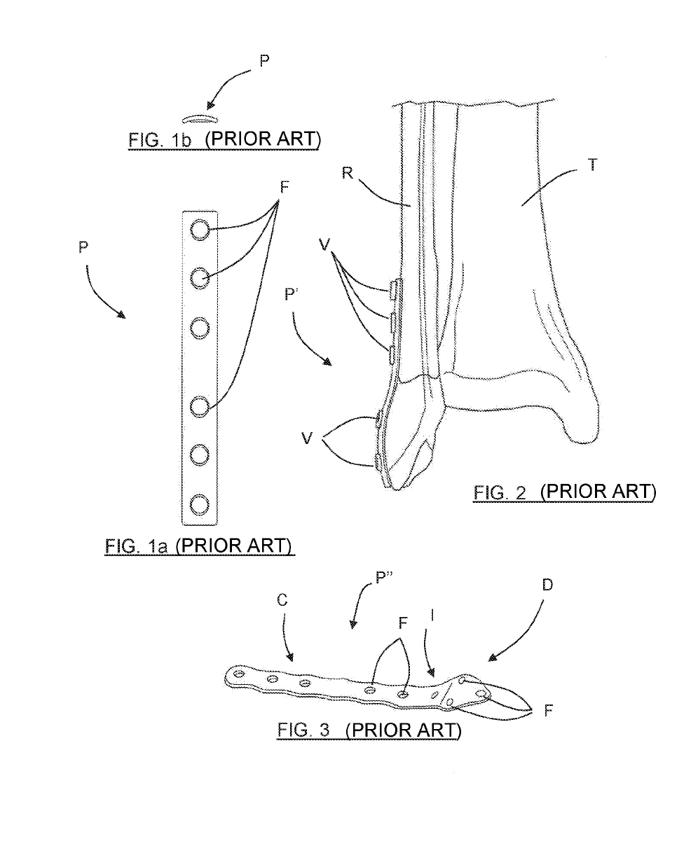

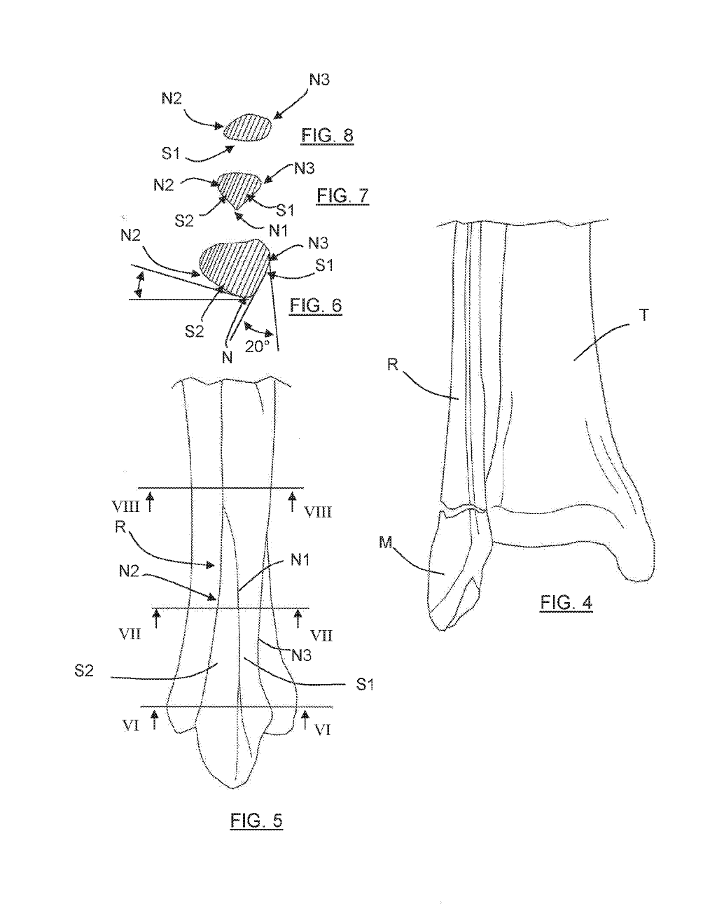

### DESCRIPTION OF PREFERRED EMBODIMENTS

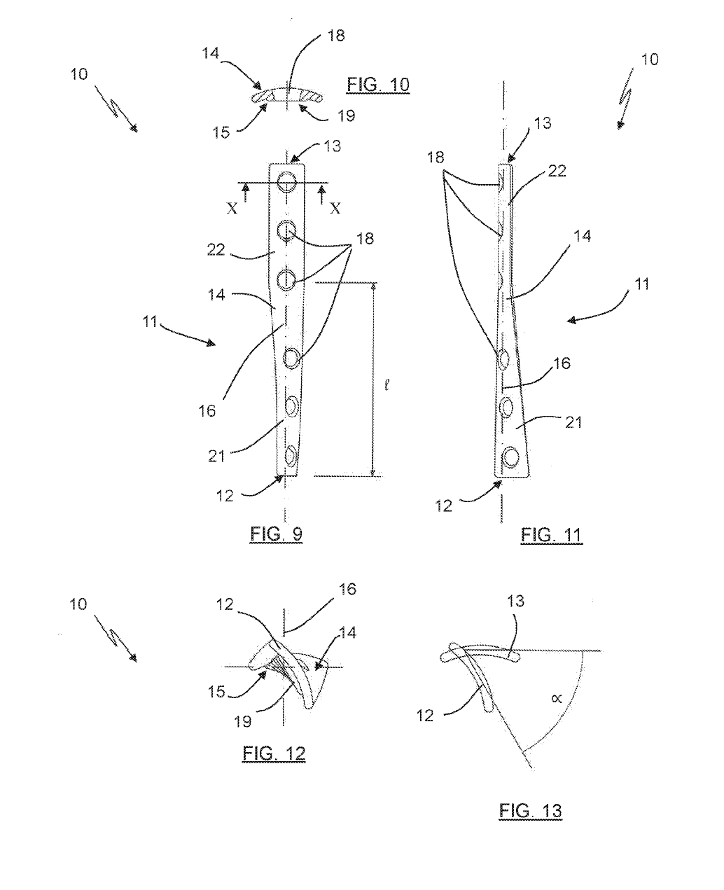

With reference to FIGS. 9, 10, 11 and 12, it is indicated as a whole with 10 an osteosynthesis plate with six holes suitable for the fixation of fractures of the distal fibula/lateral malleolus. The plate 10 comprises an elongate body, 11, provided with a distal end, 12, a proximal end, 13, an upper surface, 14, and a lower surface, 15. The elongated body 11 extends from the distal end 12 to the proximal end 13 to define a longitudinal axis, 16. The elongated body 11 comprises a plurality of through holes 18, extending from the upper surface 14 to the lower surface 15, suitable for receiving bone screws of a conventional type used for the osteosynthesis. In the specific embodiment depicted all the holes are circular and are aligned in the direction of the longitudinal axis 16 along the transverse centreline of the elongated body. Obviously, the configuration of the holes may also be very different by making use of holes according to the types and arrangements thereof described in the cited prior art or according to still other configurations. As is the case in most of the conventional osteosynthesis plates, the elongated body 11 has an arcuate profile both of the upper surface 14 and the lower surface 15 and a substantially constant thickness except in the areas of the holes 18 in correspondence of which the lower surface 15 is provided with bosses, 19, whose purpose is to increase the length of the guided portion of the head of the bone screw. In any case, both the shape in a front view and the profile of the elongated body may be different from depicted. The lower surface 15 has an external lower left profile, 30, and right profile, 31, which form bone surface contact portions.

The elongated body 11 comprises a distal portion, 21, twisted so as to faithfully match the conformation of a first face S1 of the distal end of the fibula comprising the malleolar region, and a proximal portion, 22, having a profile with a constant orientation, as it happens in conventional plates, so as to faithfully match the path of the first face S1 of the distal end of the fibula in an area above the malleolar region. In particular, thanks to the aforementioned twist, the bone contact portions 30, 31 remain in contact with the first bone face S1 for the entire length of the plate 10. In the embodiment depicted, the length, l, of the distal portion 21 is approximately 50 mm, while the overall length of the plate 10 is approximately 80 mm. In general, the length I of the twisted distal portion 21 can vary from a minimum of 30 mm to a maximum of 70 mm, depending on the size and geometry of the fibula to which the plate is to be applied. The length of the not twisted proximal portion 22 can be very variable. As shown in FIG. 13, the total twist angle, α, is equal, in the variant depicted, to 60°, while in general it can vary from 40° up to 80 ° even as a function of the geometry of the fibula. In the example shown, the orientation of the profile varies gradually of a constant factor between the distal end 12 and the opposite end of the twisted distal portion 21, however, a not constant gradient of the orientation could also be provided, to match in an even more specific way the face S1 of the fibula.

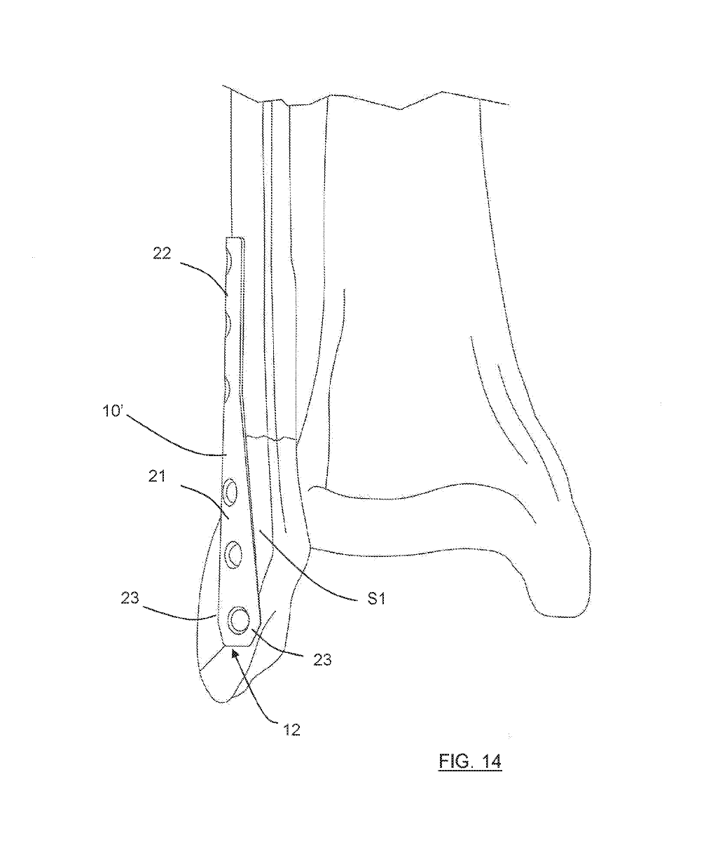

In FIG. 14, it is shown an alternative embodiment, 10′, of the plate of the invention, applied to the distal end of the right fibula, R, and in which, for simplicity of representation, the corresponding bone screws have been omitted. The embodiment 10′ includes the presence of chamfers, 23, at the two sides of the distal end 12, with a length of 2.5 mm to 5 mm from the distal end along the longitudinal side. Chamfers 23 allow an even better adaptation of the plate 10 to the geometry of the fibula.

A further advantageous variant of the plate of the invention provides a flattening of the profile at the distal end of the plate for a length of about 20 mm, and in any case comprised between 10 mm and 30 mm.

As shown in FIG. 14 an osteosynthesis plate according to the present invention when applied to the distal end of the fibula is positioned on the face S1 and runs its path without having to cross nor being superimposed at any section to the bone crest N1. Thanks to this, the bone contact portions 30 and 31 remain in contact with the bone surface over the entire length of the plate. The plate 10, 10′ is therefore in correspondence and in proximity of the distal end 12, oriented substantially posteriorly and following the shape of the face S1, so that it gradually rotates to be laterally oriented in its proximal portion 22, still facing the face S1. Besides avoiding overcoming the bone crest N1, the arrangement outlined above of the plate of the invention has additional significant advantages.

As it will be described below, in the implant operation, during the temporary stabilization of the plate on the bone, the latter tends to push forwards the distal fragment, usually gone backwards because of the fracture, and thus it facilitates the restoration of the original length and bone rotation and the tibio-peroneus-talar matching.

Since the plate 10, 10′ is positioned posteriorly in the distal region, sufferance of tissues due to the presence of the plate itself is minimized, since, compared to the lateral region, the rear region of the malleolus is “covered” (presence of soft tissue interposed between the bone surface and the skin). In fact, the first face S1 is the resting face of the peroneal tendons in the most distal region, corresponding to malleolar region, and the resting face of the peroneal muscles in the region coming just over the previous one. The plate of the invention is then supported throughout its length on the bone wall of the lateral compartment of the leg that houses the peroneal muscles and tendons. The reduced tissue suffering consequently reduces the risk of necrosis and wound dehiscence.

Furthermore, the sagittal thickness of the malleolus is higher than the transverse thickness so that it is possible to insert in the distal holes of the plate of the invention bone screws of increased length, with greater stability of positioning.

Finally, a significant advantage connected to the adoption of a plate for the osteosynthesis of malleoulus according to the present invention relates to the ability to place in one of the holes of the plate itself a syndesmosis screw. In fact, in the case of over-syndesmosis fracture with rupture of the syndesmosis, according to the conventional fixation techniques a conventional plate for osteosynthesis is applied in a lateral position with its bone screws, while a syndesmosis screw is independently applied to the rear face at syndesmosis level with substantially sagittal direction. With the use of a plate for osteosynthesis according to the invention, since the distal portion of the plate is located in the rear position, it is possible to insert the syndesmosis screw in a hole of the plate that lies at the level of the syndesmosis.

The above-mentioned advantages are still safe even in the presence of further variants and modifications to the above illustrated as examples.

In addition, the use of a plate for osteosynthesis as described above allows applying a peculiar and innovative procedure for fixation of fractures of the distal fibula/lateral malleolus.

In fact, the fracture is first recomposed by performing a general fixation, without necessarily having to perform a particularly accurate fixation. Subsequently, a temporary stabilization of a plate for osteosynthesis according to the present invention is performed on a fragment of the proximal fibula by means of a fixation clamp. Thanks to the geometry of a plate for osteosynthesis according to the present invention, the distal portion oriented substantially in the coronal plane pushes forwards the distal bone fragment helping to maintain the correct fixation of the fracture or even improve it, facilitating the restoration of the length and bone rotation and the peroneal-talar congruence. Once the osteosynthesis plate is temporary stabilized on the proximal bone fragment it is possible to check and possibly to further adjust the position of the distal bone fragment, and then proceeding to fasten the plate by inserting at least one bone screw in a hole of the plate located in correspondence of the proximal bone fragment and subsequently at least a bone screw in a hole of the plate located at a distal bone fragment. Thanks to the perfect matching between the geometry of the plate for osteosynthesis and the geometry of the bone, the insertion of the bone screws does not generate stresses that tend to mutually move the two fragments and there is not therefore the risk of a new fracture during the fastening of the plate.

These and other advantages of the plate for osteosynthesis and the method of fixation of fractures of the distal fibula/lateral malleolus of the present invention remain safe even in the presence of further modifications or variations to the above described, though always remaining within the sphere of protection defined by the claims that follow.

1.-5. (canceled)

6. An osteosynthesis plate for application to a bone surface of a distal fibula and a lateral malleolus, comprising:
an elongated body having a distal end, a proximal end, an upper surface, a lower surface determining a longitudinal axis and a plurality of holes passing from the upper surface to the lower surface disposed substantially along the longitudinal axis;
wherein the elongated body includes a distal portion geometrically twisted so as to substantially match a conformation of a first face of a distal end of the fibula oriented predominantly towards a rear direction at a malleolar level and predominantly laterally at a distal region coming just over the malleolar level, so that bone contact portions of the plate with the bone surface remain in contact with the first face for an entire length of the plate.

7. The osteosynthesis plate according to claim 6, wherein a total angle of twist between the distal end and another end of the distal portion is between 40° and 80°.

8. The osteosynthesis plate according to claim 7, wherein the twisted distal portion extends from the distal end for a length between 30 mm and 70 mm, while a proximal portion extending contiguous to it without solution of continuity of its upper and lower surfaces has a profile with a constant orientation.

9. The osteosynthesis plate according to claim 8, wherein the distal end is chamfered on both sides with a chamfer length of at least 2 mm.

10. The osteosynthesis plate according to claim 9, wherein the elongated body has a substantially constant section with a profile of the upper and lower surfaces arcuate in a transverse direction, except in a distal region of a length not exceeding 30 mm from the distal end, in which the profile of the upper and lower surfaces is substantially flat.
